(19) United States
(12) Patent Application Publication
(10) Pub. No.: US 2023/0226317 A1
(43) Pub. Date: Jul. 20, 2023

(54) STEERABLE MEDICAL DEVICE, HANDLE FOR A MEDICAL DEVICE, AND METHOD FOR OPERATING A MEDICAL DEVICE

(71) Applicant: Boston Scientific Medical Device Limited, Galway (IE)

(72) Inventors: Doran Avivi, Toronto (CA); Jan-Hung Chen, Georgetown (CA)

(21) Appl. No.: 18/189,354

(22) Filed: Mar. 24, 2023

Related U.S. Application Data
(63) Continuation of application No. PCT/IB2021/057649, filed on Aug. 19, 2021.
(60) Provisional application No. 63/084,770, filed on Sep. 29, 2020.

(51) Int. Cl. A61M 25/01

(52) U.S. Cl. CPC A61M 25/0136; A61M 25/0147

(57) ABSTRACT

A steerable medical device includes a handle having a handle body and a first control assembly that is movable with respect to the handle body. A first cam is housed within the handle body and is coupled to the control assembly. Movement of the control assembly with respect to the handle body drives rotation of the cam with respect to the handle body. A first slider is housed within the handle body and is coupled to the cam. Rotation of the cam drives translation of the slider within the handle body. An elongate tool extends from the handle. A first control wire is coupled between the first slider and the tool. Translation of the first slider causes tensioning of the control wire, and tensioning of the control wire causes deflection of the tool.

### CROSS-REFERENCE TO RELATED APPLICATIONS

This application claims the benefit of International Application Number PCT/IB2021/057649, entitled “STEERABLE MEDICAL DEVICE, HANDLE FOR A MEDICAL DEVICE, AND METHOD FOR OPERATING A MEDICAL DEVICE,” and filed Aug. 19, 2021, which claims the benefit of U.S. Provisional Application No. 63/084,770, entitled “STEERABLE MEDICAL DEVICE, HANDLE FOR A MEDICAL DEVICE, AND METHOD FOR OPERATING A MEDICAL DEVICE,” and filed Sep. 29, 2020, which are hereby incorporated by reference in their entireties.

### FIELD

This document relates to medical devices. More specifically, this document relates to steerable medical devices such as steerable sheaths, catheters, and introducers.

### SUMMARY

The following summary is intended to introduce the reader to various aspects of the detailed description, but not to define or delimit any invention.

Steerable medical devices are disclosed. According to some aspects, a steerable medical device includes a handle having a handle body and a first control assembly that is movable with respect to the handle body. A first cam is housed within the handle body and is coupled to the control assembly. Movement of the control assembly with respect to the handle body drives rotation of the cam with respect to the handle body. A first slider is housed within the handle body and is coupled to the cam. Rotation of the cam drives translation of the slider within the handle body. An elongate tool extends from the handle. A first control wire is coupled between the first slider and the tool. Translation of the first slider causes tensioning of the control wire, and tensioning of the control wire causes deflection of the tool.

In some examples, the first control assembly includes a knob assembly that is rotatable with respect to the handle body. The handle body can extend along a handle axis, and the knob assembly can be rotatable about an axis of rotation that is perpendicular to the handle axis.

In some examples, the first cam is fixed to the control assembly. In some examples, the first cam is coupled the control assembly via a gear train. The first cam can include a first spur gear, and the knob assembly can include a second spur gear engaged with the first spur gear.

In some examples, the first control assembly includes a switch assembly that is slidable with respect to the handle body. The switch assembly can include a rack, and the cam can include a pinion gear engaged with the rack.

In some examples, rotation of the cam in a first rotational direction with respect to the handle body drives translation of the first slider in a first translational direction to cause deflection of the tool in a first deflectional direction. The steerable medical device can further include a second slider housed within the handle body and coupled to the cam, and a second control wire coupled between the second slider and the tool. Rotation of the cam in a second rotational direction with respect to the handle body can drive translation of the second slider in a second translational direction, translation of the second slider can cause tensioning of the second control wire, and tensioning of the second control wire can cause deflection of the tool in a second deflectional direction.

In some examples, the tool is a sheath, a catheter, or an introducer.

Handles for medical devices are also disclosed. According to some aspects, a handle for a medical device includes a handle body and a first control assembly that is movable with respect to the handle body. A first cam is housed within the handle body and is coupled to the control assembly. Movement of the control assembly with respect to the handle body drives rotation of the cam with respect to the handle body. A first slider is housed within the handle body and is coupled to the cam. Rotation of the cam drives translation of the slider within the handle body. A first control wire is coupled to the first slider. Translation of the first slider causes tensioning of the control wire.

In some examples, the first control assembly includes a knob assembly that is rotatable with respect to the handle body. The handle body can extend along a handle axis, and the knob assembly can be rotatable about an axis of rotation that is perpendicular to the handle axis.

In some examples, the first cam is fixed to the control assembly. In some examples, the first cam is coupled the control assembly via a gear train. The first cam can include a first spur gear, and the knob assembly can include a second spur gear engaged with the first spur gear.

In some examples, the first control assembly includes a switch assembly that is slidable with respect to the handle body. The switch assembly can include a rack, and the cam can include a pinion gear engaged with the rack.

In some examples, rotation of the cam in a first rotational direction with respect to the handle body drives translation of the first slider in a first translational direction. The handle can further include a second slider housed within the handle body and coupled to the cam, and a second control wire coupled to the second slider. Rotation of the cam in a second rotational direction with respect to the handle body can drive translation of the second slider in a second translational direction, and translation of the second slider can cause tensioning of the second control wire.

Methods for operating a medical device are also disclosed. According to some aspects, a method for operating a medical device includes: a. moving a control assembly of a handle of the medical device to drive rotation of a cam housed within a body of the handle; driving translation of a first slider by rotation of the cam; tensioning a first control wire by translation of the first slider; and deflecting a tool coupled to the handle by tensioning of the first control wire.

In some examples, step a. includes rotating a knob assembly with respect to the handle body. In some examples, step a. includes transmitting rotation of the knob assembly to the cam via a gear train. In some examples, step a. includes sliding a switch assembly with respect to the handle body. In some examples, step a. includes transmitting sliding of the switch assembly to the cam via a rack and pinion assembly.

### BRIEF DESCRIPTION OF THE DRAWINGS

The accompanying drawings are for illustrating examples of articles, methods, and apparatuses of the present disclosure and are not intended to be limiting. In the drawings:

FIG. 1 is a perspective view of an example steerable medical device;

FIG. 2 is a perspective view of the interior of the handle of FIG. 1;

FIG. 3 is a top view of the interior of the handle of FIG. 1;

FIG. 4 is a top view of a portion of the interior of the handle of FIG. 1, with a cam of the handle rotated in a first rotational direction;

FIG. 5 is a top view of a portion of the interior of the handle of FIG. 1, with the cam of the handle rotated in a second rotational direction;

FIG. 6 is a perspective view of the interior of another example handle for a steerable medical device;

FIG. 7 is a perspective view of the interior of another example handle for a steerable medical device;

FIG. 8 is a perspective view of the interior of another example handle for a steerable medical device;

FIG. 9 is a perspective view of another example steerable medical device;

FIG. 10 is a perspective view of the interior of the handle of FIG. 9;

### DETAILED DESCRIPTION

Various apparatuses or processes or compositions will be described below to provide an example of an embodiment of the claimed subject matter. No example described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Generally disclosed herein are steerable medical devices that include a handle and a tool such as a sheath, a catheter, or an introducer. The handle can enable the user to manipulate or steer the tool in a desired direction. More specifically, the handle can include a control assembly (e.g. a knob assembly or a switch assembly) that is movably coupled to a housing (also referred to as a ‘body’) of the handle. In operation, movement of the control assembly can allow the user to steer or deflect the tool. The movement of the control assembly can be converted into a deflection of the tool via a cam and one or more sliders, which can be within the housing, and one or more control wires, which are coupled between the slider(s) and the tool. Movement of the control assembly can cause rotation of the cam, and rotation of the cam can cause translation of the slider(s). Translation of the slider(s) can cause a tensioning of the control wire(s), which results in a deflection of the tool. For simplicity, details of the control wires and the connection between the control wires, the tool, and the slider are not disclosed herein. However, related sliders and control wires are disclosed in, for example, U.S. Pat. No. 10,661,057 (Davies et al.), which is incorporated herein by reference in its entirety. Furthermore, steerable medical devices including sliders and control wires are sold Baylis Medical Company, Inc. (Montreal, Canada) under the brand name SureFlex® Steerable Guiding Sheath.

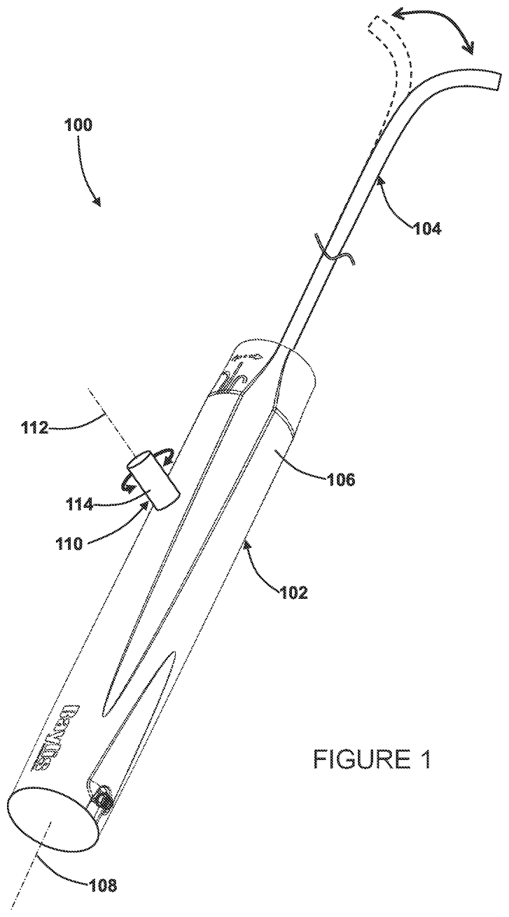

Referring now to FIG. 1, an example steerable medical device 100 is shown. The steerable medical device 100 generally includes a handle 102 and an elongate tool 104 extending from the handle. The tool 104 can be, for example (but not limited to), a sheath, a catheter, or an introducer.

Referring still to FIG. 1, the handle 102 includes a handle body 106, which extends along a handle axis 108, and a control assembly that is movable with respect to the handle body 106. In the example shown, the control assembly is in the form of a knob assembly 110 that is rotatable with respect to the handle body 106 to steer the tool 104. In the example shown, the knob assembly 110 is rotatable about an axis of rotation 112 that is perpendicular to the handle axis 108. In alternative examples, the knob assembly 110 can be rotatable about an axis of rotation that is parallel to the handle axis, or at another orientation with respect to the handle axis. In further alternative examples, the control assembly can be of another configuration, such as a switch assembly that is slidable with respect to the handle body to steer the tool (as described below).

Referring still to FIG. 1, in the example shown, rotation of the knob assembly 110 in a first rotational direction (e.g. clockwise) can cause the tool 104 to deflect in a first deflectional direction (i.e. to the configuration shown in solid line in FIG. 1), and rotation of the knob assembly 110 in a second rotational direction (e.g. counter-clockwise) can cause the tool 104 to deflect in a second deflectional direction (i.e. to the configuration shown in dotted line in FIG. 1).

In the example shown, the handle 102 includes a single control assembly. In alternative examples, the handle may include more than one control assembly (e.g. a first knob assembly, a second control knob assembly, and so on).

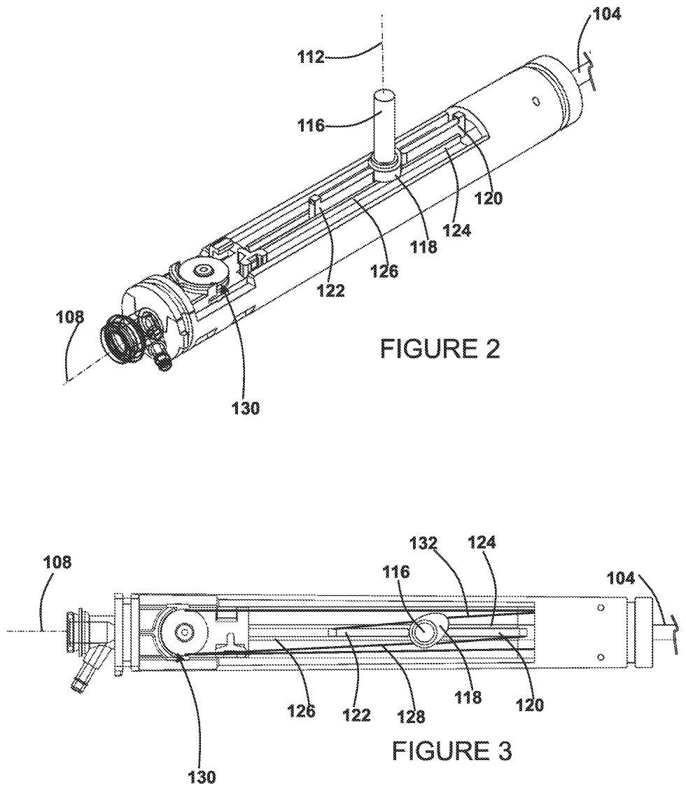

Referring now to FIGS. 2 and 3, the interior of the handle 102 is shown. In the example shown, the knob assembly 110 includes an outer knob 114 (shown in FIG. 1), which is grasped and manipulated by the user, and an inner knob 116 (shown in FIGS. 2 and 3) which is rotated by rotation of the outer knob 114.

Referring still to FIGS. 2 and 3, a cam 118 is housed within the handle body 106 (not shown in FIGS. 2 and 3), and is coupled to the control assembly so that movement of the control assembly with respect to the handle body 106 drives rotation of the cam 118 with respect to the handle body 106. More specifically, in the example shown, the cam 118 is fixed to the inner knob 116, and rotation of the knob assembly 110 about the axis of rotation 112 with respect to the handle body 106 drives rotation of the cam 118 about the axis of rotation 112 with respect to the handle body 106.

Referring still to FIGS. 2 and 3, the handle 102 further includes a first slider 120 housed within the handle body 106 and coupled to the cam 118, and a second slider 122 housed within the handle body 106 and coupled to the cam 118. The first 120 and second 122 sliders are spaced apart along the handle axis 108, and are positioned on opposite sides of the cam 118. Each slider 120, 122 is received in a respective track 124, 126, and is slidable within the track 124, 126 along the handle axis 108, towards and away from the cam 118.

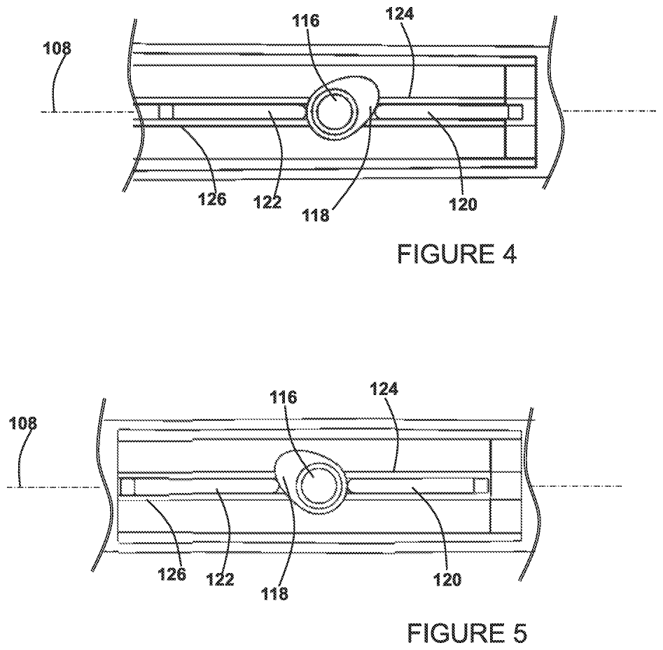

As can be seen in FIGS. 4 and 5, rotation of the cam 118 drives translation of the sliders 120, 122 within the handle body 106. That is, rotation of the cam 118 in the first rotational direction drives translation of the first slider 120 in a first translational direction (i.e. away from the cam, towards the right side of the page, as shown in in FIG. 4), and rotation of the cam 118 in the second rotational direction drives translation of the second slider 122 in a second translational direction (i.e. away from the cam, towards the left side of the page, as shown in FIG. 5).

Referring back to FIG. 3, the handle 102 further includes a first control wire 128 (shown schematically, and shown only in FIG. 3) coupled between the first slider 120 and the tool 104, via a control wire carriage assembly 130, and a second control wire 132 (shown schematically, and shown only in FIG. 3) coupled between the second slider 122 and the tool 104, via the control wire carriage assembly 130. Translation of the sliders 120, 122 causes tensioning of the control wires 128, 132, and tensioning of the control 128, 132 wires causes deflection of the tool 104. That is, translation of the first slider 120 in the first translational direction causes tensioning of the first control wire 128, which causes deflection of the tool 104 in the first deflectional direction, and translation of the second slider 122 in the second translational direction causes tensioning of the second control wire 132, which causes deflection of the tool 104 in the second deflectional direction.

The sliders 120, 122 can optionally be biased towards the cam 118 (e.g. with one or more springs), or the sliders 120, 122 and control wires can be configured so that sliding of the first slider 120 in the first translational direction causes sliding of the second slider 122 back towards the cam 118, via tension applied to the control wires 128, 132 (and vice versa).

In the example shown, the handle 102 includes a single cam 118. In alternative examples, the handle may include more than one cam (e.g. a first cam, a second cam, and so on), which can each be coupled to a respective control assembly and a respective slider. Furthermore, in the example shown, the handle 102 includes two sliders 120, 122 and two control wires 128, 132. In alternative examples, the handle can include another number of sliders and/or another number of control wires (e.g. one slider and one control wire).

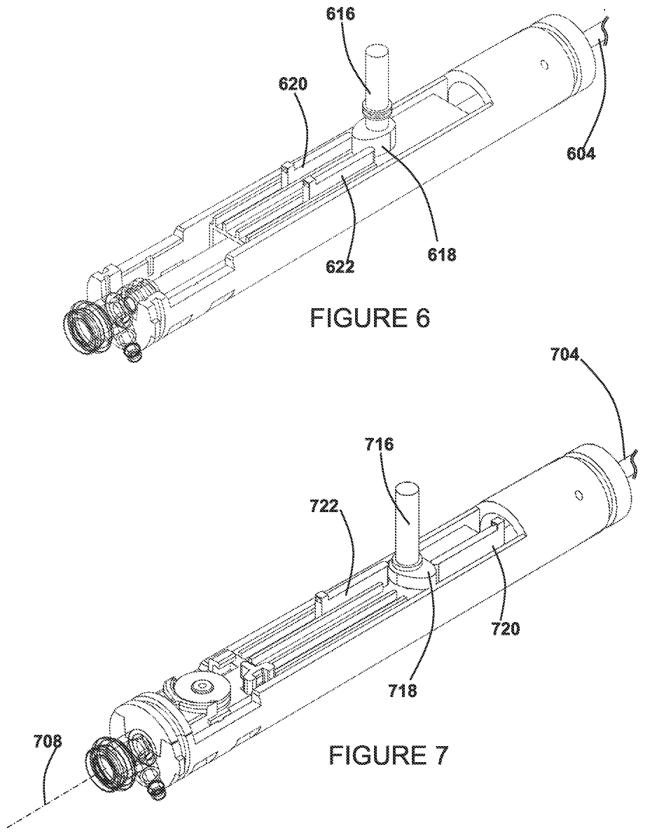

Referring now to FIGS. 6 and 7, two alternative examples of a handle are shown. In FIG. 6, features that are like those of FIGS. 1 to 5 are referred to with like reference characters, incremented by 500, and in FIG. 7, features that are like those of FIGS. 1 to 5 are referred to with like reference characters, incremented by 600. The handle of FIG. 6 is similar to the handle 102 of FIGS. 1 to 5, and includes a knob assembly (the inner knob 616 of which is shown) that drives rotation of a cam 618, and first 620 and second 622 sliders that translate in response to rotation of the cam 618 to tension control wires (not shown) and in turn cause deflection of a tool 604; however, the sliders 620 and 622 are positioned side-by-side, as opposed to in line. The handle of FIG. 7 is also similar to the handle 102 of FIGS. 1 to 5, and includes a knob assembly (the inner knob 716 of which is shown) that drives rotation of a cam 718, and first 720 and second 722 sliders that translate in response to rotation of the cam 718 to tension control wires (not shown) and in turn cause deflection of a tool 704; however, the sliders 720 and 722 are off-centre with respect to the handle axis 708.

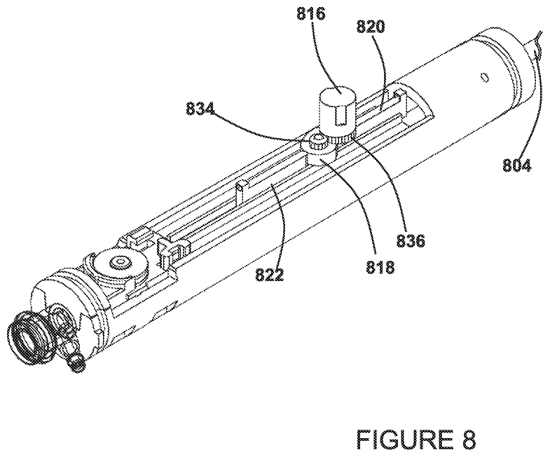

Referring now to FIG. 8, another example of a handle is shown. In FIG. 8, features that are like those of FIGS. 1 to 5 are referred to with like reference characters, incremented by 700. The handle of FIG. 8 is similar to the handle 102 of FIGS. 1 to 5, and includes a knob assembly (the inner knob 816 of which is shown) that drives rotation of a cam 818, and first 820 and second 822 sliders that translate in response to rotation of the cam 818 to tension control wires (not shown) and in turn cause deflection of a tool 804; however, in the handle of FIG. 8, the cam 818 is coupled to the control assembly via a gear train. The cam 818 includes a first spur gear 834, and the knob assembly includes a second spur gear 836 that is integral with the inner knob 816 and that is engaged with the first spur gear 834. The gear train can provide enhanced responsiveness to steering.

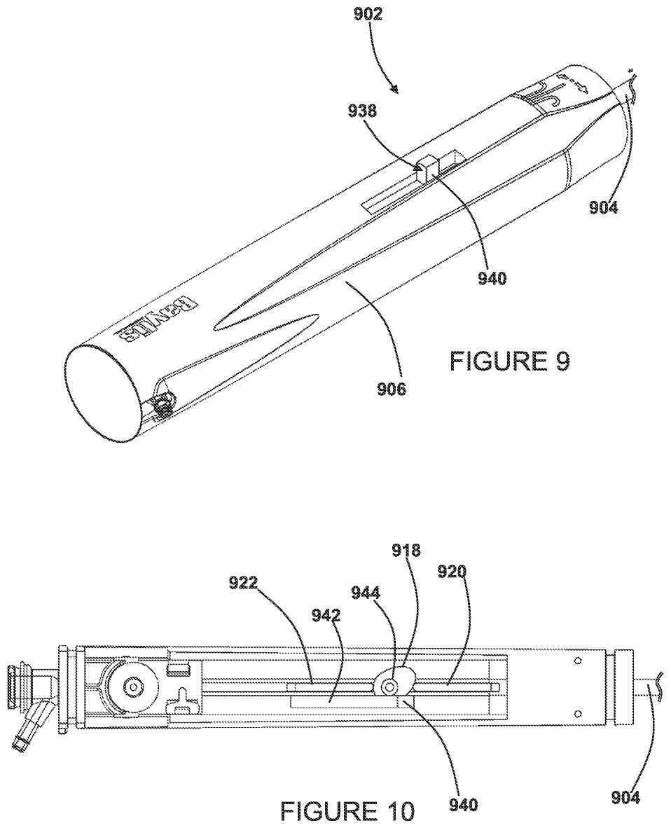

Referring now to FIGS. 9 and 10, another example of a handle is shown. In FIGS. 9 and 10, features that are like those of FIGS. 1 to 5, are referred to with like reference characters, incremented by 800. The handle 902 of FIGS. 9 and 10 is similar to the handle 102 of FIGS. 1 to 5, and includes a control assembly that drives rotation of a cam 918 (shown in FIG. 10), and first 920 and second 922 sliders (shown in FIG. 10) that translate in response to rotation of the cam 918 to tension control wires (not shown) and in turn cause deflection of a tool 904; however, in the handle of FIGS. 9 and 10, the control assembly is in the form of a switch assembly 938 that is slidable with respect to the handle body 906. The switch assembly 938 includes an outer switch 940 and a rack 942 (the teeth of which are not shown), and the cam 918 includes a pinion gear 944 (the teeth of which are not shown) engaged with the rack 942. Sliding of the outer switch 940 causes translation of the rack 942, which causes rotation of the pinion gear 944, and rotation of the cam 918.

In general, in use of the medical devices described herein, a user can move the control assembly with respect to the handle body (e.g. can rotate the knob assembly 110 or can slide the switch assembly 938), which drives rotation of the cam (optionally via a gear train or a rack and pinion assembly), which drives translation of the first slider and/or second slider, which causes tensioning of the first control wire or second control wire, which in turn causes deflection the tool.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A steerable medical device comprising:
a handle having a handle body and a first control assembly that is movable with respect to the handle body;
a first cam housed within the handle body and coupled to the first control assembly, wherein movement of the first control assembly with respect to the handle body drives rotation of the first cam with respect to the handle body;
a first slider housed within the handle body and coupled to the first cam, wherein rotation of the first cam drives translation of the first slider within the handle body;
an elongate tool extending from the handle; and
a first control wire coupled between the first slider and the tool, whereby translation of the first slider causes tensioning of the first control wire, and tensioning of the first control wire causes deflection of the tool.

2. The steerable medical device of claim 1, wherein the first control assembly comprises a knob assembly that is rotatable with respect to the handle body.

3. The steerable medical device of claim 2, wherein the handle body extends along a handle axis, and the knob assembly is rotatable about an axis of rotation that is perpendicular to the handle axis.

4. The steerable medical device of claim 2, wherein the first cam is fixed to the knob assembly.

5. The steerable medical device of claim 2, wherein the first cam is coupled the first control assembly via a gear train.

6. The steerable medical device of claim 5, wherein the first cam comprises a first spur gear, and the knob assembly comprises a second spur gear engaged with the first spur gear.

7. The steerable medical device of claim 1, wherein the first control assembly comprises a switch assembly that is slidable with respect to the handle body.

8. The steerable medical device of claim 7, wherein the switch assembly comprises a rack, and the first cam comprises a pinion gear engaged with the rack.

9. The steerable medical device of claim 1, wherein:
rotation of the cam in a first rotational direction with respect to the handle body drives translation of the first slider in a first translational direction to cause deflection of the tool in a first deflectional direction;
the steerable medical device further comprises a second slider housed within the handle body and coupled to the first cam, and a second control wire coupled between the second slider and the tool, whereby rotation of the first cam in a second rotational direction with respect to the handle body drives translation of the second slider in a second translational direction, and translation of the second slider causes tensioning of the second control wire, and tensioning of the second control wire causes deflection of the tool in a second deflectional direction.

10. The steerable medical device of claim 1, wherein the tool is a sheath, a catheter, or an introducer.

11. A handle for a medical device, the handle comprising:
a handle body and a first control assembly that is movable with respect to the handle body;
a first cam housed within the handle body and coupled to the first control assembly, wherein movement of the first control assembly with respect to the handle body drives rotation of the first cam with respect to the handle body;
a first slider housed within the handle body and coupled to the first cam, wherein rotation of the first cam drives translation of the first slider within the handle body; and
a first control wire coupled to the first slider, whereby translation of the first slider causes tensioning of the first control wire.

12. The handle of claim 11, wherein the first control assembly comprises a knob assembly that is rotatable with respect to the handle body.

13. The handle of claim 12, wherein the handle body extends along a handle body axis, and the knob assembly is rotatable about an axis of rotation that is perpendicular to the handle body axis.

14. The handle of claim 12, wherein the first cam is fixed to the knob assembly.

15. The handle of claim 12, wherein the first cam is coupled the first control assembly via a gear train.

16. The handle of claim 15, wherein the first cam comprises a first spur gear, and the knob assembly comprises a second spur gear engaged with the first spur gear.

17. The handle of claim 11, wherein the first control assembly comprises a switch assembly that is slidable with respect to the handle body.

18. The handle of claim 17, wherein the switch assembly comprises a rack, and the first cam comprises a pinion gear engaged with the rack.

19. The handle of claim 11, wherein:
rotation of the first cam in a first rotational direction with respect to the handle body drives translation of the first slider in a first translational direction;
the handle further comprises a second slider housed within the handle body and coupled to the first cam, and a second control wire coupled to the second slider, whereby rotation of the cam in a second rotational direction with respect to the handle body drives translation of the second slider in a second translational direction, and translation of the second slider causes tensioning of the second control wire.

20. A method for operating a medical device, comprising:
a. moving a control assembly of a handle of the medical device to drive rotation of a cam housed within a body of the handle;
b. driving translation of a first slider by rotation of the cam;
c. tensioning a first control wire by translation of the first slider; and
d. deflecting a tool coupled to the handle by tensioning of the first control wire.
